(19) United States
(12) Patent Application Publication
(10) Pub. No.: US 2016/0184046 A1
(43) Pub. Date: Jun. 30, 2016

(54) SURGICAL TOOL LIGHTS

(71) Applicant: Spinal Elements, Inc., Carlsbad, CA (US)

(72) Inventors: Jason Blain, Encinitas, CA (US); David Ortiz, San Diego, CA (US); Joseph Clark, San Diego, CA (US); Dean Johnson, Solano Beach, CA (US)

(21) Appl. No.: 14/979,262

(22) Filed: Dec. 22, 2015

Related U.S. Application Data
(60) Provisional application No. 62/126,244, filed on Feb. 27, 2015.
(60) Provisional application No. 62/096,283, filed on Dec. 23, 2014.

(51) Int. Cl. A61B 90/30; A61B 17/16; A61B 17/88; A61B 17/3211; A61B 17/3205; A61B 17/00; A61B 17/29

(52) U.S. Cl. CPC A61B 90/30; A61B 17/00234; A61B 17/1604; A61B 17/1655; A61B 17/1659; A61B 17/29; A61B 17/3211; A61B 17/32053; A61B 17/8875; A61B 17/3205; A61B 17/16; A61B 2090/309

(57) ABSTRACT

Surgical tools having a tool portion, a handle and a light are disclosed. The tool portion has a first connector that couples to a second connector on the handle. The light can illuminate the working end of the surgical tool. The light can be energized when the tool portion and the handle are coupled. A plurality of different types of tool portions can be coupled to the handle. Methods of using surgical tools having a light are also disclosed.

### CROSS-REFERENCE TO RELATED APPLICATIONS

The present application claims priority benefit under 35 U.S.C. §119(e) of U.S. Provisional Application Nos. 62/096,283, filed Dec. 23, 2014 and 62/126,244, filed Feb. 27, 2015, the contents of which are incorporated by reference herein in their entireties.

### BACKGROUND

1. Field

The present application relates generally to surgical tools, and more particularly to surgical tools with lights and methods of using the lighted tools for surgical procedures.

2. Background

Surgical procedures can be performed through an open incision or a minimally invasive procedure. For example, in spinal surgery, implants are placed in the intervertebral space through an open procedure using retractors. The size of the incision and the amount that the tissue is retracted is preferably minimized to reduce scarring and recovery time. In addition, minimally invasive surgical techniques have been used to access the surgical site through small incisions. Minimally invasive techniques involve accessing the surgical site through a cannula or access tube placed through a small incision to the surgical site. Minimally invasive surgery offers multiple advantages, such as minimal tissue damage, minimal blood loss, smaller incisions and scars, minimal post-operative discomfort, and relative quick recovery time and return to normal function.

The small openings used in open procedures and the small cannulas used in minimally invasive techniques, however, can make visualization of the surgical site difficult. The narrow passageways to the surgical site can block the overhead lights of the operating room. In addition, the depth of the implant site can also obscure illumination of the surgical site.

Current surgical lights used in operating rooms are bulky and/or cumbersome. Overhead lights are commonly used, but are bulky and the light is easily obstructed by the medical personnel or other tools. Some surgeons wear lights mounted to their heads, but these lights are uncomfortable and can be difficult to aim into the surgical site. Surgical light devices have been used near the surgical site to illuminate directly into the incision. However, these surgical lights take up limited space in the small incisions. Furthermore, these lights are usually tethered to a light source, which can take up limited space in the operating room. The tethered cable can also be an obstruction and cause a hazard in the operating room. Therefore, a need still exists for an easier to use and improved apparatuses and methods for providing light to a surgical site.

### INCORPORATION BY REFERENCE

All publications and patent applications mentioned in this specification are herein incorporated by reference to the same extent as if each individual publication or patent application was specifically and individually indicated to be incorporated by reference.

### SUMMARY

An aspect of at least one of the embodiments disclosed herein includes a surgical tool having a tool portion with a tool tip at a distal end and a first connector at a proximal end. The surgical tool also includes a handle with a power source and a second connector, the second connector releasably connectable to the first connector. A light source is configured to direct light toward the tool tip. The first connector can be configured to make an electrical connection with the second connector so that the power source is in electrical communication with the light source.

The light source can be disposed toward the proximal end of the tool portion. In some embodiments, the light source can include a light emitting diode. In some embodiments, the light source includes a coating on the tool portion that illuminates. The light source can be autoclavable.

In some embodiments, the surgical tool includes a locking mechanism between the first connector and the second connector.

In some embodiments, the power source is a battery that is disposed in an internal compartment of the handle. The battery can be wirelessly rechargeable. The handle can include a switch for energizing the light source.

The tool tip can be an awl, curette, screw driver, drill, tap, scalpel, ronguer, forceps, rasp, or implant holder.

In some embodiments, the tool portion includes an annular ring configured to be illuminated by the light source. In some embodiments, the light source is retractable into a cavity in the tool portion. The light source can be movable along a longitudinal length of the surgical tool. The light source can be movable around a perimeter of the surgical tool. In some embodiments, the light source is detachable from the surgical tool. The surgical tool can have more than one light source.

An aspect of at least one of the embodiments disclosed herein includes a surgical tool including a first portion with a tool tip at a distal end and a first connector at a proximal end and a second portion with a second connector at a distal end. A light can be connected to the first portion. The first connector can be releasably connectable to the second connector to provide illumination to the light.

In some embodiments, the first connector and the second connector are electrical connectors and the second portion is configured to provide electrical power to the light. In some embodiments, the first connector and the second connector include light-transmitting windows and the second portion is configured to provide a light source to the light.

The second portion can be a handle comprising a battery. The battery can be wirelessly rechargeable.

In some embodiments, the second portion is electrically connected to a power source secured to a user. The second portion can include a switch for energizing the light. The light can include a light emitting diode. In some embodiments, at least the light is autoclavable.

In some embodiments, the tool tip is made of a translucent material and the light is configured to be transmitted through the tool tip. The first portion can further include an annular ring configured to be illuminated by the light.

In some embodiments, the light is retractable into a cavity in the first portion. The position of the light can be movable on the surgical tool. The surgical tool can include more than one light.

An aspect of at least one of the embodiments disclosed herein includes a method of using a surgical tool, the method including providing a tool portion and a handle, the tool portion including a tool tip at a distal end, a light source, and a first connector at a proximal end, and the handle including a power source and a second connector. The method can include connecting the tool portion to the handle such that the first connector makes an electrical connection with the second connector. The method can include delivering the tool tip to a surgical site and illuminating the surgical site with the light source.

The light source can be a light emitting diode. In some embodiments, at least the light source is autoclavable.

The power source can be a battery that is disposed in an internal compartment of the handle.

The tool tip can be an awl, curette, screw driver, drill, tap, scalpel, ronguer, forceps, rasp, or implant holder. In some embodiments, the method further comprises retracting the light source into a cavity in the tool portion.

An aspect of at least one of the embodiments disclosed herein includes a method of using a surgical tool, the method including providing a tool portion and a handle. The tool portion can include a tool tip at a distal end, a light and a first connector at a proximal end. The handle can include a second connector. The method can include connecting the tool portion to the handle such that the first connector makes a connection with the second connector. The method can further include delivering the tool tip to a surgical site and illuminating the surgical site with the light.

In some embodiments, the first connector and second connector include light- transmitting windows and the handle is configured to provide a light source to the light. The handle can include a battery for powering the light.

In some embodiments, the method further includes adjusting the position of the light. The method can further include retracting the light source into a cavity in the tool portion.

### BRIEF DESCRIPTION OF THE DRAWINGS

These and other features, aspects and advantages of the described embodiments are described with reference to drawings of certain embodiments, which are intended to illustrate, but not to limit. It is to be understood that the attached drawings are for the purpose of illustrating concepts of the described embodiments and may not be to scale.

FIG. 1 is a rear perspective view of a surgical tool with light, according to an embodiment of the present disclosure.

FIG. 2 is a front perspective view of the surgical tool of FIG. 1.

FIG. 3 is a close-up perspective view of the light of the surgical tool of FIG. 1.

FIG. 4 is a rear perspective view of a tool component of the surgical tool of FIG. 1.

FIG. 5 is a front perspective view of the tool component of the surgical tool of FIG. 1.

FIG. 6 is a rear perspective view of a handle of the surgical tool of FIG. 1.

FIG. 7 is a front perspective view of the handle of the surgical tool of FIG. 1.

FIG. 8 is an exploded rear perspective view of the handle of FIG. 1.

FIG. 9 is a side cross-sectional view of the surgical tool of FIG. 1.

FIG. 10 is a close-up of a side cross-sectional view of the surgical tool of FIG. 1.

FIG. 11 is a front perspective view of a surgical tool with light, according to another embodiment of the present disclosure.

FIG. 12 is a side view of a surgical tool with light, according to another embodiment of the present disclosure.

FIG. 13 is a perspective view of a surgical tool with a removable light module, according to another embodiment of the present disclosure.

FIG. 14 is a perspective view of a surgical tool with a movable light, according to another embodiment of the present disclosure.

FIG. 15A is a perspective view of a tool portion with a light-transmitting window, according to another embodiment of the present disclosure.

FIG. 15B is a perspective view of a handle with a light-transmitting window, according to another embodiment of the present disclosure.

### DETAILED DESCRIPTION

As will be explained herein, certain embodiments of surgical tools with lights provide advantages over the prior art devices. For example, the surgical tools with lights disclosed herein can provide improved illumination of the surgical site while minimizing obstruction of the surgical site.

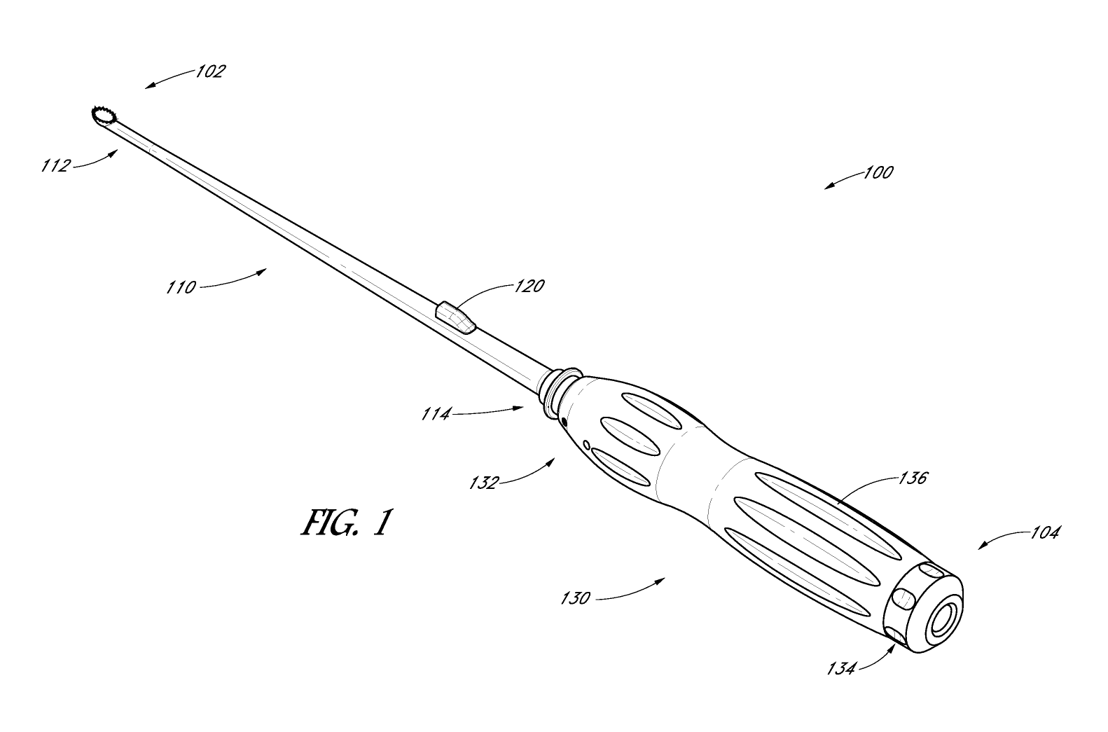

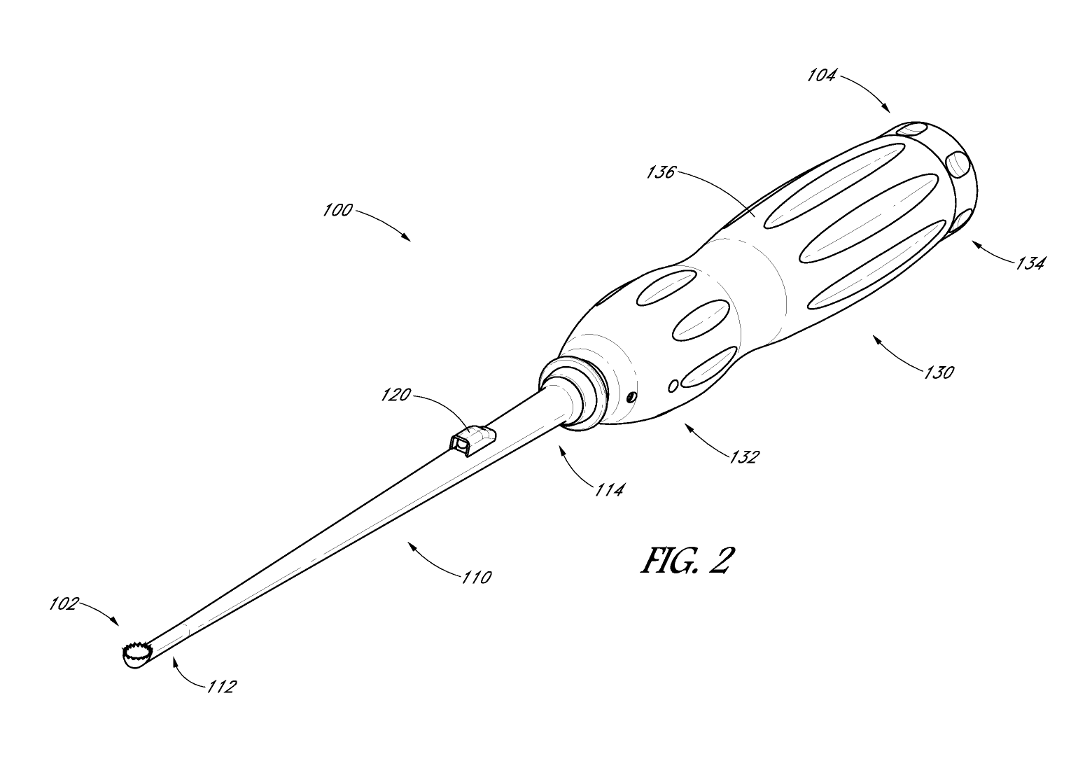

FIGS. 1 and 2 illustrate an example of a surgical tool 100 with a light. The surgical tool 100 includes a distal end 102 with a tool portion 110 and a proximal end 104 with a handle 130. The tool portion 110 has a first end 112, which can have any of a plurality of different types of tools, such as an awl, curette, screw driver, drill, tap, scalpel, ronguer, forceps, rasp, cauterizer, implant holder, and the like. A curette is shown in the embodiments illustrated in the figures. The tool portion 110 can have an elongate portion between the first end 112 and a second end 114. The elongate portion can be configured to extend through an incision to the surgical site. The second end 114 of the tool portion 110 can be attached to a first end 132 of the handle 130. The handle 130 can have a first end 132, a second end 134 and a grip portion 136 between the first and second ends. The grip portion 136 can have a rubberized and/or textured surface to aid in gripping the handle 130.

The surgical tool 100 has a light 120 that can be integrated with the tool, as in the illustrated embodiments. As illustrated in FIGS. 1-3, the light 120 can be disposed on the tool portion 110 toward the second end 114 of the tool portion 110. Positioning the light 120 toward the second end 114 advantageously distances the heat potentially produced from the light away from the surgical site, preferably outside of the incision in the patient's skin. Furthermore, the distance of the light 120 from the distal end 102 of the surgical tool 100 can allow for a wider illumination area of the light. In addition, positioning the light 120 toward the second end 114 can help prevent the light from interfering with other equipment in the surgical access space, and from injuring the anatomical structures of the patient. In cases where the surgical tool is used through a cannula, such as in minimally invasive procedures, the light can be configured to be positioned outside of the cannula to prevent interference with the cannula walls and to help prevent occluding visualization of the surgical site. In some embodiments, the light can be disposed on the handle. For example, the light can be at or near the first end 132 of the handle 130 directed toward the distal end 102 of the surgical tool 100.

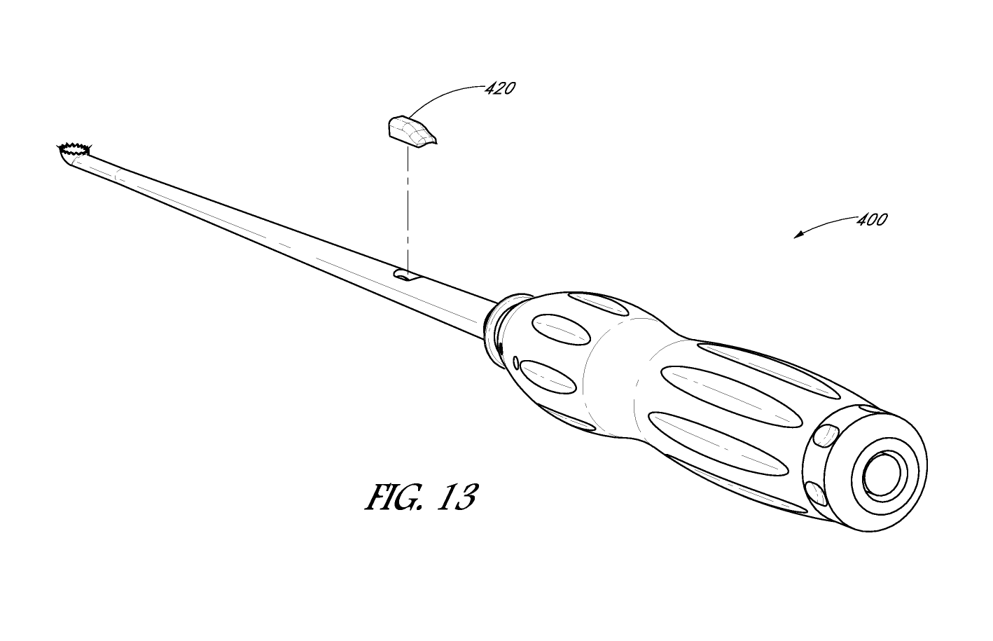

Although the light is described in some embodiments as being integrated with the surgical tool, in other embodiments, a separate light module 420 can be attached to the surgical tool 400, as illustrated in FIG. 13. The light module 420 can be attached to the surgical tool with adhesives, clamps, fasteners and the like. In some embodiments, the light module can be attached to a collar that is mounted to the shaft of the surgical tool. In some embodiments, the light module can be a separate component with a first attachment mechanism that connects with a complementary second attachment mechanism on the surgical tool. For example, the light module can have hooks that attach to notches on the surgical tool. In another example, the light module can have a threaded shaft that fastens into a threaded hole in the surgical tool. The light module can be portable and contain its own light source and power source, such that it can be attached to existing surgical tools without modification of the tools. In other embodiments, the light module can have an electrical connector or a light-transmitting window that couples with a complementary electrical connector or light-transmitting window on the surgical tool.

In some embodiments, the light module is exchangeable such that different types of lights can be attached to the surgical tool, depending on the situation. For example, an ultraviolet light can be used in combination with equipment for detecting the ultraviolet light in order to visualize the surgical site. In some embodiments, multiple lights are attached to a surgical tool for increased light intensity and/or wider light distribution.

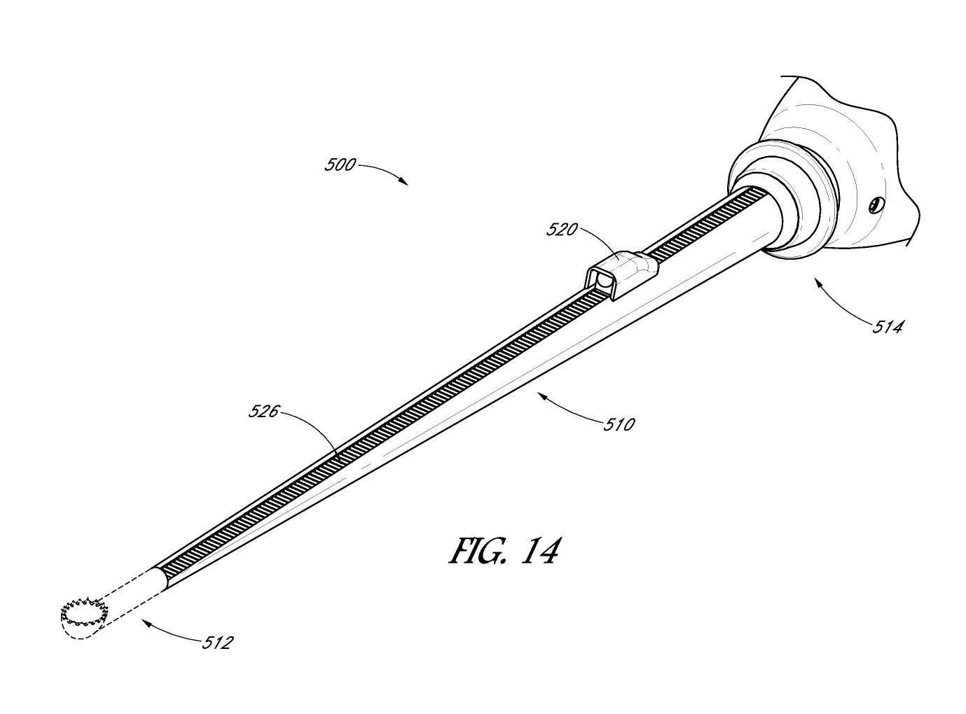

With reference to FIG. 14, in some embodiments, the position of the light 520 is adjustable. The light 520 can be moved longitudinally along the shaft of the tool portion 510, and/or laterally around the perimeter or circumference of the tool portion 510. For example, the tool portion 510 can have a track 526 extending along its longitudinal length and the light 520 can be adjustable along the track 526 so that the light 520 can move distally toward the first end 512 or proximally toward the second end 514. In some embodiments, the track 526 can include electrical contacts extending along its length to provide power to the light 520. In some embodiments, the light 520 can be connected to a retractable cable that delivers electrical power or illumination. In another example, the light can be attached to a sleeve that rotates around the circumference of the tool portion. A movable light advantageously allows adjustment of the light intensity and the focus by changing the position of the light. Also, being able to change the position of the light can help avoid obstacles that can block the light beam, such as other surgical equipment and the patient's anatomy.

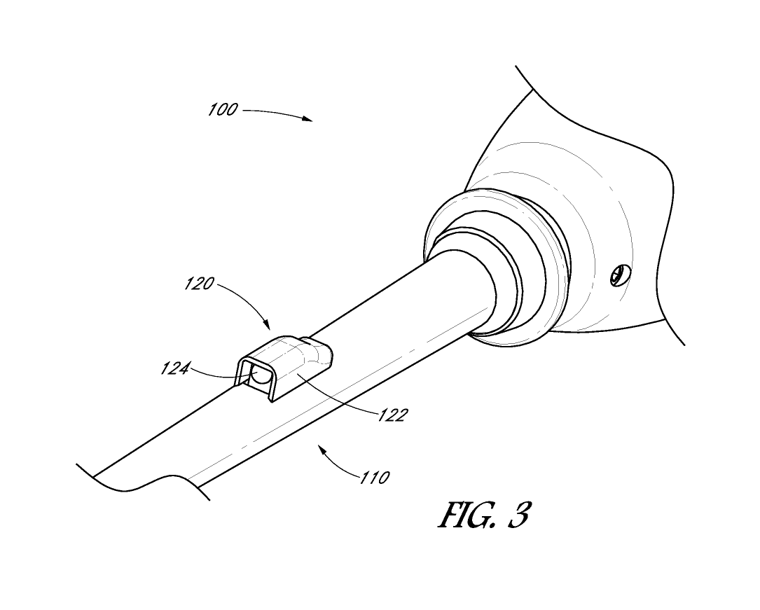

With reference to FIG. 3, the light 120 can include a light housing 122 that encompasses the light source 124. In some embodiments, the light housing 122 contains and seals the light source 124 from contamination. In other embodiments, the light housing 122 covers without sealing the light source 124. The light housing 122 can help protect the light source 124 from contamination, such as from the patient's blood and bodily fluids, as well as the harsh conditions associated with sterilization, such as during autoclaving. The light housing 122 can also direct the light toward the working end, i.e., the distal end 102 of the surgical tool 100, such as with reflectors. In some embodiments, the light housing 122 is translucent to allow passage of the light from the light source to the surrounding environment.

The light source 124 can have any of a plurality of different types of light producing devices. For example, the light source 124 can be a light emitting diode (LED), an incandescent bulb, a halogen bulb, a fluorescent bulb, a laser, an electroluminescent material, or other functional light source. The light source preferably produces no heat or negligible heat to reduce the likelihood of injury to the surrounding tissue. In some embodiments, the light housing 122 is insulated to minimize heat transfer from the light source 124 to the surrounding tissue. In some embodiments, the light source 124 is connected to a heat sink to dissipate the heat. For example, the body of the tool portion itself can act as a heat sink.

By positioning the light source on or near the tool portion, it advantageously allows for brighter illumination of the surgical site. The light does not have to be transmitted a long distance, or only transmitted a short distance so that there is little to no loss of light intensity. The light is produced at the surgical tool and directly illuminates the surgical site, instead of the light being produced by a separate machine and then transmitted to the surgical site, such as through a fiber optic cable.

Furthermore, while the light can be spread over a wide area by placing the light source a distance from the distal end 102, as discussed above, it can also be focused to provide increased intensity of light. The LED or other light source can be inherently focused, or can be focused using reflectors, or can be focused by adjusting the position of the light source. In contrast, other lighting means can scatter the light and result in a light spot with less intensity.

The light source is preferably autoclaveable so that the light source can be cleaned and sterilized along with the rest of the surgical tool. In some embodiments, the light source can be embedded in epoxy or sealed in an enclosure. For example, the light source can be sealed in the light housing and shine through a transparent window.

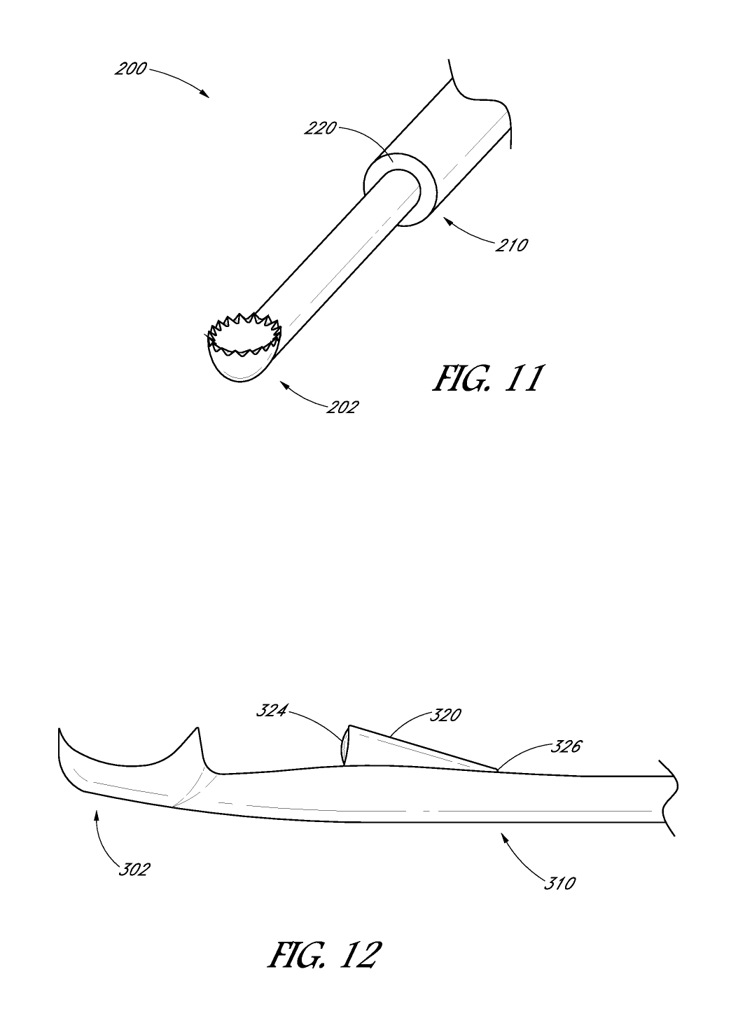

In some embodiments, the light can be retractable into the surgical tool. For example, the light housing can have a hinge toward its proximal end and the distal end can pivot up to expose the light source, or pivot down to reduce the profile of the tool when a light is not needed or for use in limited spaces, as discussed further in FIG. 12.

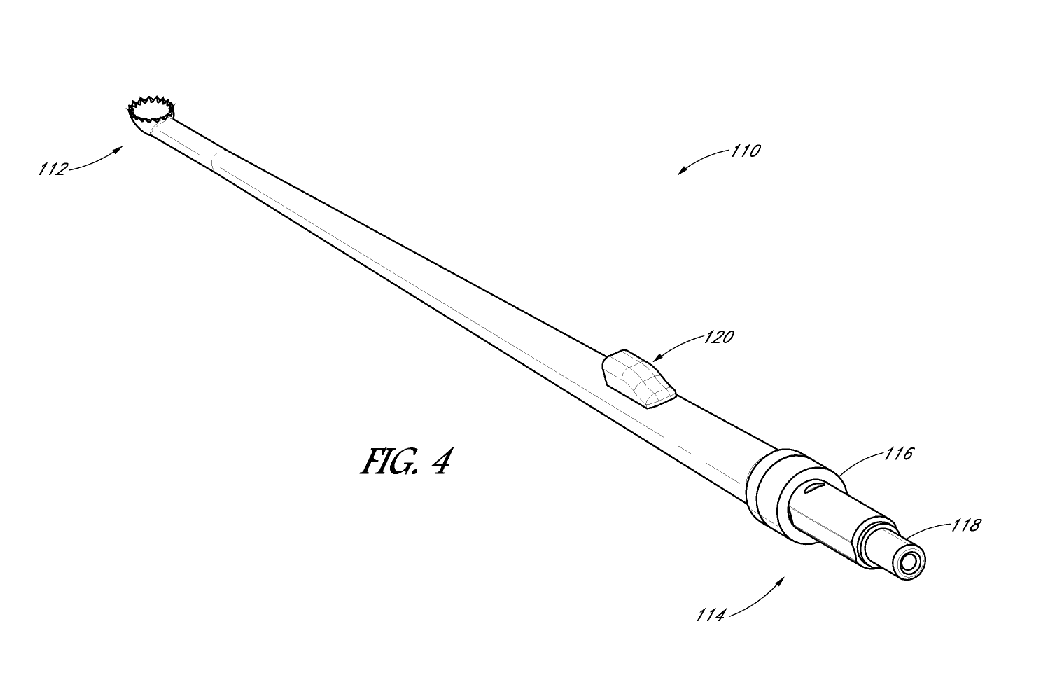

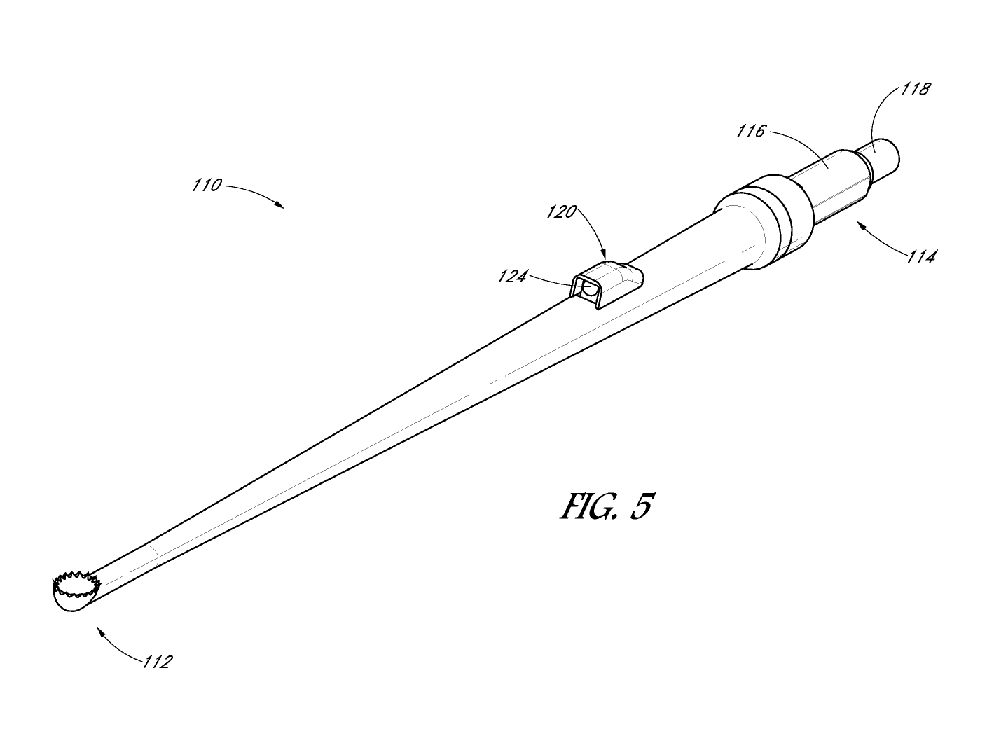

With reference to FIGS. 4 and 5, the tool portion 110 includes a first end 112 and a second end 114. As mentioned above, the first end 112 can be any of a plurality of different types of tools, such as an awl, curette, screw driver, drill, tap, scalpel, ronguer, forceps, rasp, cauterizer, implant holder, and the like. The second end 114 of the tool portion 110 can be configured to attach to a first end 132 of the handle 130. A first coupling 116 at the second end 114 can be configured to engage with a complementary second coupling on the handle 130. The illustrated embodiment shows a quick connect first coupling with detents or cavities that can engage with balls or protrusions on the quick connect second coupling in order to secure the tool portion 110 to the handle 130.

The second end 114 of the tool portion 110 can also include a first electrical connector 118 configured to be releasably connected with a complementary second electrical connector on the handle 130. In the illustrated embodiments, the first electrical connector 118 is a cylindrical male plug configured to couple with a female plug in the handle 130. The first electrical connector 118 is in electrical communication through the shaft with the light source 124 to deliver power to the light. In some embodiments, the first electrical connector couples with the second electrical connector automatically when the first coupling is connected to the second coupling. In other embodiments, the first electrical connector couples with the second electrical connector separately from the first coupling and second coupling.

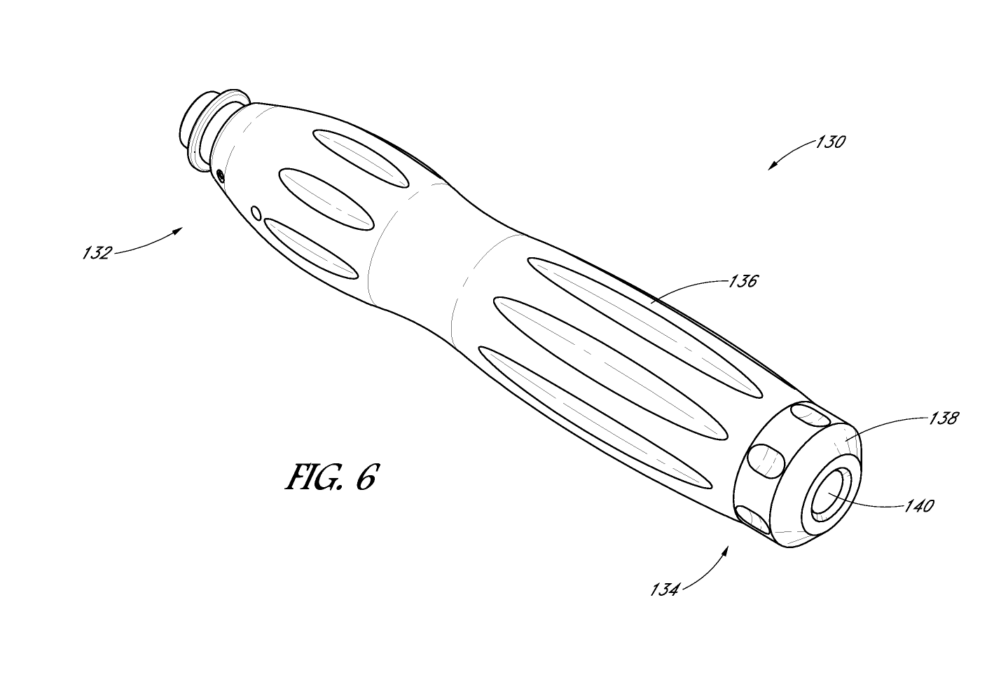

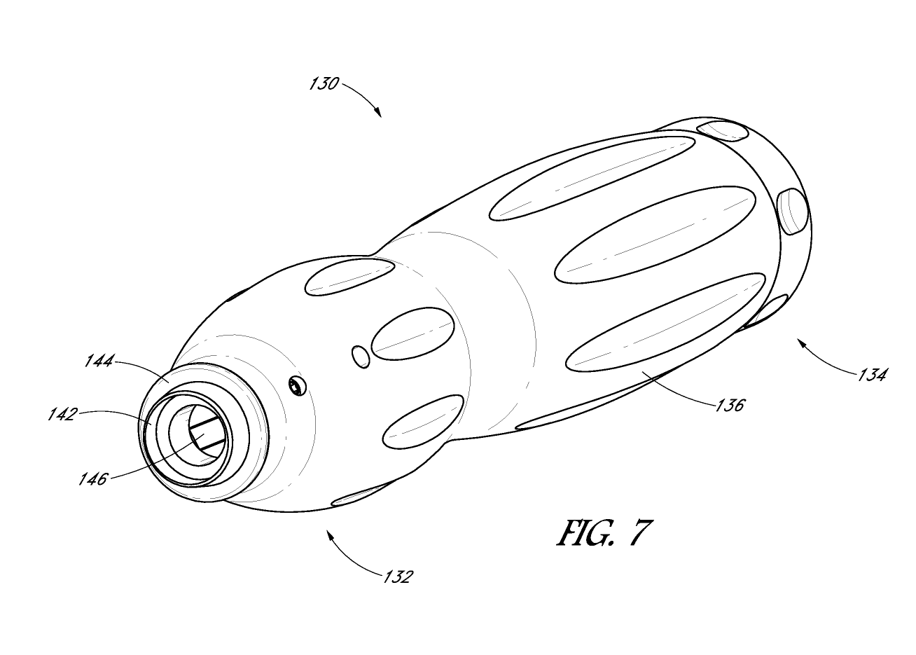

With reference to FIGS. 6 and 7, the handle 130 can include a first end 132, a second end 134 and a grip portion 136 therebetween. The grip portion 136 can have a rubberized and/or textured surface to aid in gripping the handle 130. In some embodiments, the handle 130 has an endcap 138 toward the second end 134 that can be removed to provide access to the internal compartment of the handle 130. The second end 134 can also have a switch 140 to turn the light on and off, and in some embodiments to dim the light. In the illustrated embodiment, the switch 140 is a push button toggle disposed in the center of the endcap 138. In other embodiments, the switch can be located at other positions on the handle, such as the grip portion or the first end of the handle. In some embodiments, the switch can be any of a plurality of different types of switches, such as for example a rotatable knob integrated with the endcap that turns the light on and off, and in some embodiments dims the light.

As shown in FIG. 7, the first end 132 of the handle 130 can have a second coupling 142 configured to engage with the first coupling 116 on the tool portion 110. The second coupling 142 can be any of a plurality of different connector designs. In the illustrated embodiment, the second coupling 142 is a quick connect design with a protrusion projecting from the inner surface that engages with a cavity on the outer surface of the first coupling 116. A locking mechanism 144 can be disposed on the second coupling 142 or first coupling 116 to lock the two connectors together and prevent the second coupling 142 from inadvertently disengaging from the first coupling 116. In other embodiments, the first coupling and the second coupling can have any functional coupling, such as for example screw connections, quarter-turn connections, ratcheting connections, and the like.

The first end 132 of the handle 130 can include a second electrical connector 146 that is configured to releasably connect with the first electrical connector 118 on the tool portion 110. In the illustrated embodiment, the second electrical connector 146 is a female plug that couples with the male plug on the tool portion 110. In other embodiments, the electrical connectors can be any functional electrical coupling that can transmit electrical power, such as for example a universal serial bus (USB) connection or a pin and socket connection. The second electrical connector 146 is in electrical communication with a battery or other functional power source. When the first coupling 116 is connected to the second coupling 142, the first electrical connector 118 can also connect with the second electrical connector 146, such that both mechanical and electrical connections are achieved with a single operation. In other embodiments, the mechanical connection can be performed separately from the electrical connection.

FIG. 8 is an exploded view of an embodiment of the handle 130. The handle 130 can include a second coupling 142 and a locking mechanism 144 attached to an end of the grip portion 136, which is configured to couple with the first coupling 116 of the tool portion 110. A second electrical connector 146 can be attached toward the end of the grip portion 136 and configured to electrically couple with the first electrical connector 118 of the tool portion 110. As in the illustrated embodiment, the second coupling 142 and second electrical connector 146 can be coaxial such that the mechanical connection and electrical connection are achieved in the same operation as the tool portion 110 is coupled to the handle 130.

A battery 148 or other power source can be electrically connected to the second electrical connector 146 and disposed in the internal compartment of the handle 130. In some embodiments, the electrical power from the battery 148 can be regulated by circuitry disposed between the battery 148 and the second electrical connector 146. For example, a constant current driver and/or voltage regulator can be used to condition the power signal delivered to the light source.

With continued reference to FIG. 8, the handle can also include a switch 140 electrically coupled to the battery 148 and configured to provide power or interrupt power to the light source to turn the light on and off, and in some embodiments to dim the light. The switch 140 can be a pushbutton switch, a lever, a rotational switch, or any functional switch. An endcap 138 can be attached to the grip portion 136 to hold the internal components of the handle 130. The illustrated endcap 138 is a screw type cap fastened to the second end of the handle 130. In other embodiments, the endcap can have a press fit connection, a hook connection, or any functional connection.

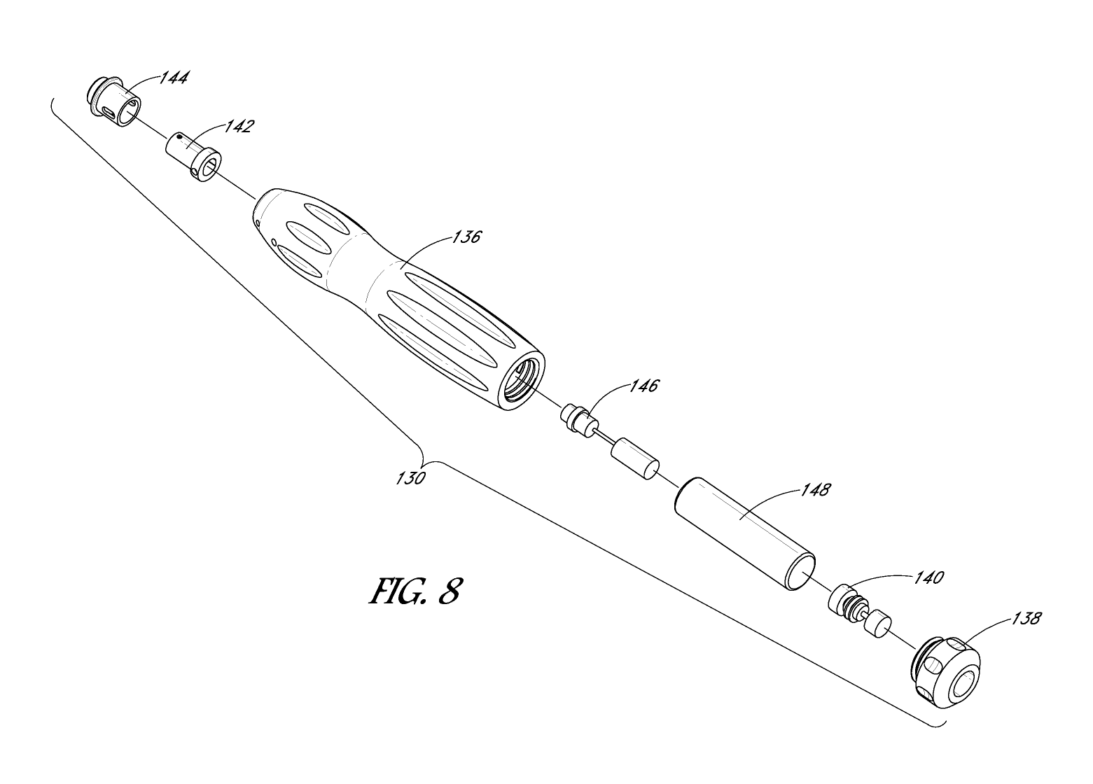

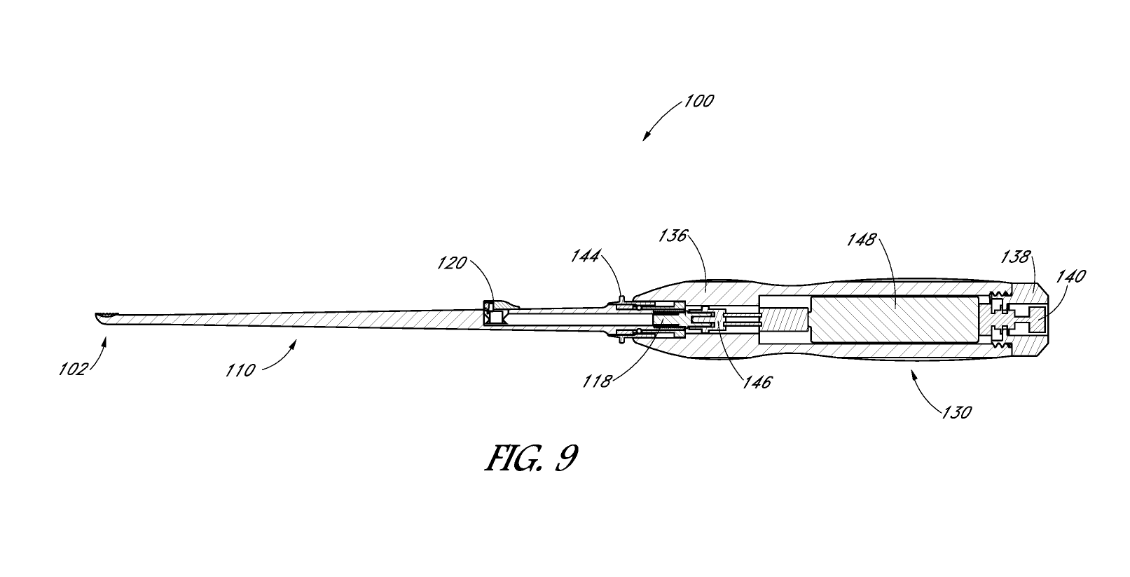

FIGS. 9 and 10 are cross-sectional views of the surgical tool 100 with the tool portion 110 connected to the handle 130. In the illustrated embodiment, the handle 130 can have an internal compartment which houses the battery 148, second electrical connector 146 and second coupling 142. The first coupling 116 with the first electrical connector 118 is engaged with the second coupling 142 with the second electrical connector 146. When the switch 140 is activated, a circuit is completed and the battery 148 sends electrical power through the second electrical connector and first electrical connector to the light 120 to illuminate the distal end 102 of the surgical tool 100.

The surgical tool disclosed herein provides several advantages over other surgical tools. For example, the present surgical tool positions the light source (e.g., LED) near the work area, which results in brighter illumination of the work area with minimal light loss. The light source, particularly an LED, produces a bright light that can be focused to illuminate a specific area. Other surgical tools transport light from a remote light source, which can result in reduced light intensity from light loss during the transmission. The light transmission often produces a scattered light pattern that is not focused. Furthermore, the light transmission components can obstruct the view through the working channel to the surgical site.

Being able to focus the light advantageously enables the light to be placed a distance away from the work area and the patient while still providing a focused light. The light can be positioned at a distance so that any heat produced from the light source does not damage or adversely affect the patient's tissue. For example, the position of the light on the surgical tool can be configured so that the light is outside the incision when in use and the light can shine through the incision to the work area. In some embodiments, the light is adjustable and can be focused to shine through the incision to the work area.

In some embodiments, the surgical tool is also portable and does not need to be tethered to other equipment in the operating room. The power source and light source can be self-contained in the surgical tool, which eliminates the need for an external connection to other equipment. Having a self-contained power source eliminate wires or cables running to the surgical tool, which may interfere with the surgeon's mobility around the surgical site.

Another advantage of the surgical tool disclosed herein is the modular tool portion and handle. The tool portion and handle can be quickly and easily interchanged for use with multiple tool tips, or for mid-surgery recovery, such as in case of tool malfunction. For example, the tool portion and handle have a single connection that couples the mechanical coupling as well as the electrical connection, which makes the interchanging process quick and easy. When different tools are needed during surgery, the user can replace the tool portion while using the same handle and power source. Also, when the battery in the handle is discharged or malfunctions, the handle can be easily and quickly replaced.

Furthermore, another advantage of the current disclosure is in the weight balance, which can be similar to traditional tools that do not have a light. The battery, which is typically the heaviest component of the surgical tool, is disposed in the handle, which can result in the center of mass being in or near the handle. Having the center of mass near the handle can help make the surgical tool easy to manipulate and control.

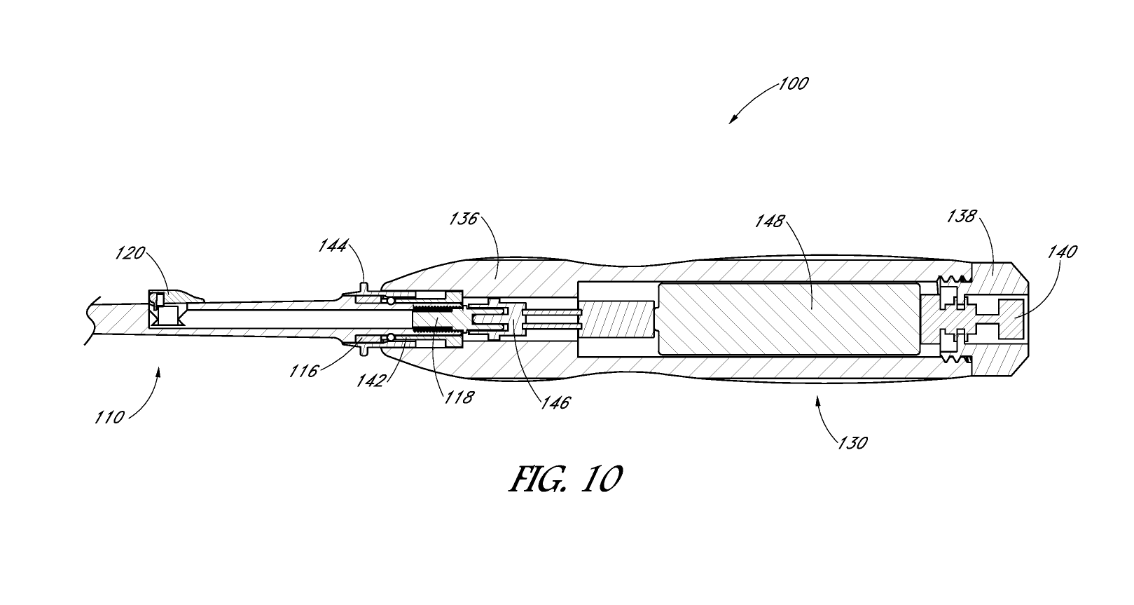

Other configurations of a surgical tool with a light on the tool are also envisaged. For example, with reference to FIG. 11, the tool portion 210 can have an annular ring 220 facing the distal end 202 of the surgical tool 200 that can be illuminated by a light source that is in the surgical tool. In some embodiments, the annular ring portion can be made of a translucent material that directs the light from the light source out through the annular ring toward the distal end. The light source can be housed or embedded in the translucent material.

In some embodiments, the surgical tool has multiple lights. The lights can be positioned in any suitable location, such as around the circumference of the tool portion, at various locations along the longitudinal length of the tool, and/or any location on the handle. The lights can be positioned on different portions of the surgical tool for improved illumination of the surgical site. Each light or group of lights can have independent on/off controls to illuminate different portions of the surgical tool and surgical site, depending on the situation. In another example, electrical connection strips are disposed along or around the tool portion. The lights can be positioned at any location along the electrical connection strips such that the lights are in electrical communication with the strips. The strips can be energized to illuminate the lights.

Some parts of the surgical tool can be made of a translucent material to transmit light. For example, the surgical tool can be a scalpel with a blade made of a translucent material, such as acrylics, glass, ceramics, plastics, and the like. The light source is in luminous communication with the blade, such as adjacent to the blade or proximal to the blade and connected to the blade through a fiber optic cable. In some embodiments, only portions of the blade may be translucent while other portions are opaque or reflective.

In some embodiments, portions of the surgical tool can be covered in a material that changes light transmission properties with the application of an electrical charge, light or heat (e.g., electrochromic, photochromic, thermochromic, suspended particle, liquid crystal material). The surgical tool can be made of a translucent material and illuminated as discussed above. The material's opacity can be changed to allow more or less light through from the translucent material. Different portions of the surgical tool can be controlled independently to adjust light intensity and direction of light projection.

In some embodiments, the surgical tool can have a paint, epoxy, or other coating that illuminates, without excitation or when an electrical charge, light, or heat is applied (i.e., electroluminescent, photoluminescent, thermoluminescent). For example, the surgical tool can be a suture needle that is coated with a glowing paint to make the needle and the area around the needle easier to see while suturing tissue. In another example, the surgical tool can be coated in a luminescent paint that illuminates when an ultraviolet light is applied.

In some embodiments, the surgical tool can have a cavity inside a translucent shell and luminescent fluid can be delivered to the cavity to light up the tool tip or other portions of the surgical tool. The fluid can be a chemiluminescent fluid, electroluminescent fluid, or any of a plurality of different types of luminescent fluids.

In another example, the surgical tool can be connected to a portable power source that is worn on, or positioned near the surgeon or an operating room assistant. For example, the surgeon can have a battery pack worn on his hip with an electrical lead connected to a surgical tool. In some embodiments, the electrical lead can be interchangeable with several different surgical tools. In some embodiments, the same handle can be interchangeable with several different tool portions, as discussed above. These embodiments advantageously reduce the weight on the handle from the absence of the power source in the handle, which can minimize fatigue on the surgeon.

Furthermore, the portable power source can advantageously be in the sterile field of the operating room so that the power source can be handled and manipulated by the surgeon without risking contamination from outside the sterile field. The sterile field is the area of the operating room where the equipment and personnel have been sterilized, and non-sterilized items from outside the sterile field are prohibited. The power source can be controlled, or replaced in case of malfunction, without breaking the sterile field. In contrast, many current surgical light systems are powered by a power source that is bulky and positioned outside the sterile field during surgery. The surgeon is not able to manipulate the non-sterilized power source in current systems without breaking the sterile field.

FIG. 12 illustrates another configuration of a surgical tool 300 having a light 320. The light 320 is disposed near the distal end 302 of the tool portion 310. Positioning the light 320 close to the distal end 302 can advantageously provide a focused light that is not obstructed by other instruments or tissue in the surgical channel. In some embodiments, the light 320 can have a hinge 326 and the light can pivot into the tool portion 310 when the light is not being used, in order to provide improved visualization of the surgical site.

In some embodiments, the surgical tool can include controls for manipulating the light. For example, the tool can have controls to increase or decrease the light intensity. In some embodiments, the tool can have controls for focusing the area of the light and/or changing the direction of the light. For example, the light housing can be on a swivel to direct the light in various directions.

The light can be any of a plurality of different colors or wavelengths. For example, the light can be white, blue, red, infrared, ultraviolet or x-ray. The different colors of light may be advantageous for helping to distinguish certain tools, implants, or anatomy. The different wavelength lights can be used with detection equipment to help visualize the surgical site. For example, the light can be an infrared light that illuminates the surgical site for visualization by an infrared camera.

In some embodiments, the surgical tool can be a tissue retractor with the light disposed on or near the retractor blades. A light housing can be disposed on one or more of the retractor blades and configured to illuminate the retracted surgical site. A power source can be disposed on the retractor frame, carried by the surgeon or assistant, or on a separate device, to power the one or more lights, as discussed above. In some embodiments, separate power sources can be disposed on each of the retractor blades to power each respective light.

In some embodiments, the retractor blades can be made of a translucent material and the light can be transmitted through the blades to the surgical site. For example, the light source can be external of the retractor blades, such as in the retractor frame or a separate device, and the light can be transmitted through fiber optic cables to the one or more translucent retractor blades that emit the light onto the surgical site. Some parts of the blade can be opaque or reflective, while some parts can be translucent to direct or focus the light toward the surgical site.

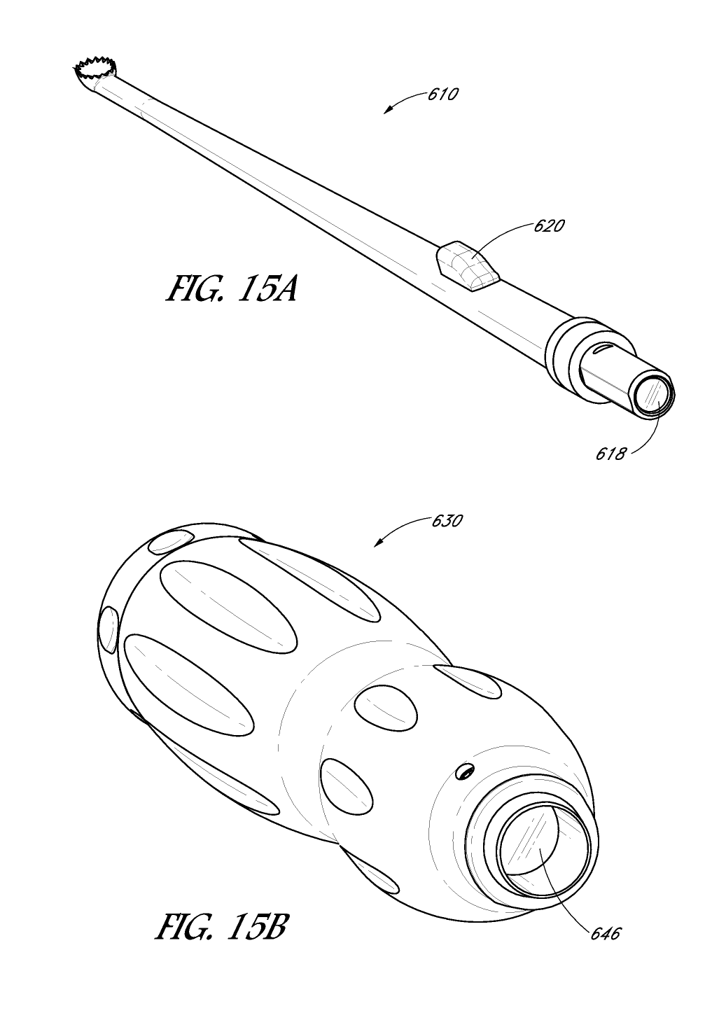

In some embodiments, instead of an electrical connector between the handle and tool portion, a fiber optic connection can be disposed between the handle 630 and the tool portion 610, as illustrated in FIGS. 15A-B. The fiber optic connection can comprise a first light-transmitting window 618 at the end of the fiber optic cable on the tool portion 610 and a second light-transmitting window 646 at the end of a fiber optic cable on the handle 630. The light is transmitted through the fiber optic connection when the light passes out from the second light-transmitting window 646 of the handle 630 into the adjacently positioned first light-transmitting window 618 on the tool portion 610. The light can then be transmitted through the tool portion 610 and projected out the light module 620. The light module 620 can have reflectors that direct the light to the working end of the tool. The light source can be disposed within the handle 630 or an external device and the light transmitted from the handle 630 to the tool portion 610 through the fiber optic connection.

In some embodiments, the power source can be a rechargeable battery. The rechargeable battery can be in the handle or other location as discussed above. For example, the rechargeable battery can be disposed in an attachable light module discussed above, and the light module can be removed from the surgical tool for charging. Several light modules can be used during a surgical procedure for different types of lights or in case the battery charge is expended.

The battery can be charged through a wired connection, or through a wireless charging method, such as induction charging, radio wave charging, and the like. In some embodiments, a wireless charging tray or mat accommodates a set of several surgical tools that can all be charged simultaneously. During surgery, the surgical tools can be placed on the charging tray or mat to recharge the instruments after use. Preferably, a light, meter, or other indicator is disposed on the surgical tool that shows the level of charge of the battery.

In a method of use, a surgical tool is provided. In embodiments with a separate power source, the power source is connected to the surgical tool. For example, the power source can be a battery pack worn by the user, or a stand-alone battery pack, and an electrical lead from the battery pack is connected to the surgical tool. In some embodiments, the power source is in the handle of the surgical tool and the handle can be connected to the tool portion. In some embodiments, the surgical tool is on a wireless charging tray being charged until the tool is picked up for use.

The surgical tool is introduced into the incision and through the working channel to the surgical site. In some embodiments, a minimally invasive procedure is used and the surgical procedure is performed through a cannula. Visibility of the surgical site through the cannula can be difficult and the lighted surgical tool can help to illuminate through the cannula for improved visualization of the surgical site. It may be useful to adjust the light to have a tight focus of light for working through a narrow cannula. In other embodiments, the surgery can be an open procedure and the light can have a wider spread to illuminate a large portion of the surgical site.

The light can be turned on before introducing the surgical tool into the incision or after the surgical tool reaches the surgical site. The switch is preferably on or near the handle so that the light can be turned on or off after the surgical tool is introduced to the surgical site. In some embodiments, the light is adjusted during surgery to focus the light on the surgical site. An adjustment mechanism can be on the handle to easily manipulate the focus of the light during surgery. Once the surgical site is sufficiently illuminated, the surgical tool is used to perform an orthopedic procedure. The procedure can involve cutting, fastening, hole-forming, tapping, scraping, grabbing, implanting, cauterizing, suturing, or the like using a tool portion having an awl, curette, screw driver, drill, tap, scalpel, rongeur, forceps, rasp, cauterizer, needle, implant holder, or other device.

For example, the surgical tool can be an inserter tool that holds a device, such as an intervertebral implant, for implanting in the patient. The inserter tool can have a mechanism for coupling with the implant, such as clamps or a threaded shaft. First, the implant site can be prepared to accept the implant using one or more other surgical tools, such as curettes, rasps, drills, etc. Then, the implant can be inserted using the inserter tool. The inserter tool can have one or more lights that are configured to shine on the surgical site. In some embodiments, the positions of the lights are adjustable so that the light can be aimed around the implant to the implant site.

In some embodiments, a first tool portion is used for a first procedure on the surgical site and then the first tool portion is removed from the handle. A second tool portion is connected to the handle and a second procedure is performed. The second tool portion can also include a light that may be actuated from the handle to illuminate the surgical site. The light on the second tool portion can also be adjustable to manipulate the focus of the light. Other tool portions, or previously used tool portions, can be used with the handle to perform additional procedures until the entire surgery is completed.

In some embodiments, more than one handle can be included in a kit so that multiple handles can be connected with the handles at the same time. Having more than one handle can reduce surgery times by not having to change tool portions during surgery, or by having an assistant change tool portions on one handle while the surgeon uses another handle. In some embodiments, the handles can be rechargeable and some handles can be recharged while other handles are in use. In some embodiments, the surgical tool with light is disposable and may be discarded after use or when the battery is depleted.

The surgical tools with lights described herein can be used in orthopedic surgical procedures, such as spinal surgery, hip surgery, arthroplasty, and the like. In some embodiments, the surgical tool with lights can be used in other surgical procedures, such as dental surgeries, cardiac surgeries, vascular surgeries, neurosurgeries, etc.

While certain embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A surgical tool comprising:
a first portion comprising a tool tip at a distal end and a first connector at a proximal end;
a second portion comprising a second connector at a distal end;
a light module configured to provide light toward the tool tip;
wherein the first connector is releasably connectable to the second connector to illuminate the light module.

2. The surgical tool of claim 1, wherein the first connector and the second connector are electrical connectors and the second portion is configured to provide electrical power to the light module.

3. The surgical tool of claim 1, wherein the first connector and the second connector comprise light-transmitting windows and the second portion is configured to provide a light source to the light module.

4. The surgical tool of claim 1, wherein the second portion is a handle comprising a battery.

5. The surgical tool of claim 4, wherein the battery is wirelessly rechargeable.

6. The surgical tool of claim 1, wherein the second portion is electrically connected to a power source secured to a user.

7. The surgical tool of claim 1, wherein the first portion comprises a coating that illuminates.

8. The surgical tool of claim 1, wherein the light module comprises a light emitting diode.

9. The surgical tool of claim 1, wherein at least the light module is autoclavable.

10. The surgical tool of claim 1, wherein the tool tip is an awl, curette, screw driver, drill, tap, scalpel, ronguer, forceps, rasp, or implant holder.

11. The surgical tool of claim 1, wherein the tool tip is made of a translucent material and the light is configured to be transmitted through the tool tip.

12. The surgical tool of claim 1, wherein the first portion further comprises an annular ring configured to be illuminated by the light.

13. The surgical tool of claim 1, wherein the light module is retractable into a cavity in the first portion.

14. The surgical tool of claim 1, wherein the light module is movable along a longitudinal length of the surgical tool.

15. The surgical tool of claim 1, wherein the light module is movable around a perimeter of the surgical tool.

16. The surgical tool of claim 1, wherein the light module is detachable from the surgical tool.

17. The surgical tool of claim 1, further comprising more than one light module.

18. A method of using a surgical tool, the method comprising:
providing a tool portion and a handle;
the tool portion comprising a tool tip at a distal end, a light module and a first connector at a proximal end; and
the handle comprising a second connector;
connecting the tool portion to the handle such that the first connector makes a connection with the second connector;
delivering the tool tip to a surgical site; and
illuminating the surgical site with the light.

19. The method of claim 18, wherein the first connector makes an electrical connection with the second connector for providing electrical power to the light module.

20. The method of claim 18, wherein the first connector and second connector comprise light-transmitting windows and the handle is configured to provide a light source to the light module.

21. The method of claim 18, wherein the handle comprises a battery for powering the light module.

22. The method of claim 18, further comprising adjusting the position of the light module.

23. The method of claim 18, further comprising retracting the light module into a cavity in the tool portion.
